(19) United States
(12) Patent Application Publication
(10) Pub. No.: US 2025/0171834 A1
(43) Pub. Date: May 29, 2025

(54) IN SITU NUCLEIC ACID ANALYSIS USING PROBE PAIR LIGATION

(71) Applicant: The Johns Hopkins University, Baltimore, MD (US)

(72) Inventors: Harry Larman, Baltimore, MD (US); Joel Credle, Gaithersburg, MD (US)

(21) Appl. No.: 18/972,277

(22) PCT Filed: Jun. 7, 2023
(86) PCT No.: PCT/US2023/024735

§ 371 Date: Dec. 6, 2024

(51) Int. Cl. C12Q 1/6841; C12Q 1/6855; C12Q 1/6876

(52) U.S. Cl. CPC C12Q 1/6841; C12Q 1/6855; C12Q 1/6876

(57) ABSTRACT

In one aspect, compositions are provided for the simultaneous in situ quantification and localization of RNA sequences with subcellular precision are utilized in the methods. In particular, specific hybridization of at least one probe set in a sample is followed by in situ ligation, which locks specifically circularized probe set around an RNA target sequence. Rolling circle amplification followed by fluorescently labeled detector probe hybridization, enables simultaneous in situ quantification and localization of RNA sequences with subcellular precision.

The present application claims the benefit of U.S. Provisional Application No. 63/349,787 filed Jun. 7, 2022, which is incorporated herein by reference in its entirety.

### FIELD

The present disclosure relates to the field of ribonucleotide analysis. More specifically, the present disclosure provides compositions and methods for in situ RNA analysis enabling simultaneous in situ detection and localization of RNA sequences with subcellular precision.

### BACKGROUND

Traditional gene expression methods that use bulk RNA analysis lack the ability to resolve transcript location, and thus fail to capture the inherent cellular heterogeneity present in virtually every tissue. Abundance and localization of RNA transcripts mapped to specific regions of tissue can complement histological analysis, providing an additional layer of molecular information¹. Subcellular resolution of mRNA abundance and location can in some cases be used to categorize biologically important cell-to-cell variability and interactions²⁻⁴.

The tumor microenvironment (TME) describes the cellular composition surrounding solid tumors with a specific focus on the immune cell composition in this particular region of interest. Antibody-based methods (immunofluorescence-THC, immunohistochemistry-IF) measure specific protein expression profiles that help to determine the immune cell composition of the TME. While useful, IHC and IF analyses are often limited in specificity (due to antibody cross-reactivity) and are challenging to multiplex⁵. Technologies that alternatively quantify RNA in situ have therefore emerged as complementary to antibody-based assays. In situ detection of RNA within the TME can provide highly multiplexed measurements with spatial precision. Also, several diseases have disruptions of mRNA localization as a defining feature (e.g., spinal muscular atrophy, amyotrophic lateral sclerosis); it is thus necessary to develop improved methods that capture RNA abundance with spatial resolution^{6,7}.

Several methods and technology platforms have been developed to measure both RNA abundance and spatial location in situ. Fluorescence In Situ Hybridization (FISH) and derivatives of this technique, which use serial reprobing (e.g., seqFISH, MERFISH), while promising, have failed to move beyond certain technical limitations such as multiplexing limits due to molecular crowding (<hundred targets measured), the need for dedicated imaging platforms with low sample throughput and high costs (>40 probes required per transcript)⁸⁻¹¹. In situ sequencing (e.g., FISSEQ, Bar-Seq) of RNA molecules, like FISH-based methods, also suffer from many of the same technical challenges^{12,13}. scRNA-seq based methods (e.g., Drop-seq, Slide-seq) can profile whole transcriptomes, yet at high per sample cost (>$10,000) due to sequencing depth requirements combined with the computational difficulty in linking transcriptomes back to cellular locations prevents these methods from being widely adopted¹⁴⁻¹⁶. In these techniques, spatial resolution is determined by pixel size, which may not be of sufficient resolution (e.g., single cell) for certain applications. Recently shown to be a robust highly multiplexed method for detection of RNA, LISH (Ligation In situ Hybridization) is a probe ligation-based technology that has not yet been adopted for use in measuring both the abundance and transcript position in situ.¹⁸

### SUMMARY

The present disclosure is based, at least in part, on the improvement of a multiplexed probe ligation method termed “LISH-Lock'n'Roll.”

In particular embodiments, adjacent hybridization of probes in a sample is followed by in situ ligation that locks a specifically circularized probe set around an RNA target sequence. Rolling circle amplification (“Lock'n'Roll”), followed by fluorescently labeled detector probe hybridization, enables simultaneous in situ quantification and localization of RNA sequences with subcellular precision.

Accordingly, the present disclosure provides compositions and methods for detecting nucleic acids. In one aspect, the present disclosure provides compositions and methods for detecting ribonucleic acids. In another aspect, the present disclosure can be used to detect deoxyribonucleic acids. It is understood that embodiments reciting the detection of RNA is applicable to the detection of DNA.

In certain aspects, a method of detecting and/or localizing target nucleic acid sequences (e.g. target RNA sequences) in situ, comprising: contacting a biological sample comprising a target DNA sequence (e.g. target DNA sequence) with a composition comprising (i) a target-specific ligation probe set and hemi-bridge oligonucleotides, (ii) at least one ligase and (iii) a strand-displacing polymerase; incubating the reaction mixture of step (a) under conditions that permit hybridization of the at least one ligation probe set to the target sequence present in the biological sample; circularizing the probe set and amplifying the circularized probe by strand-displacement amplification; and detecting and localizing target DNA sequences (e.g. target RNA sequences) in situ. In certain embodiments, the strand displacing DNA polymerase comprises Phi29 polymerase or Bst polymerase.

In certain embodiments, the hemi-bridge oligonucleotides and the ligation probe set are annealed prior to contacting the biological sample. In certain embodiments, the hemi-bridge oligonucleotides may be protected from exonuclease degradation by protecting 3′ ends using such means as adding phosphorothiated linkages in the last 3 bases at the 3′ end of the bridge or ligation probes, for example.

In additional aspects, methods for provided for detecting and/or and localizing target nucleic acid sequences (e.g. target RNA sequences) in situ, comprising: (a) contacting a biological sample comprising a target nucleic sequence (e./g. RNA sequence) with a composition comprising (i) a target-specific ligation probe set and an oligonucleotide primer that binds to one ligation probe, (ii) at least one double strand DNA or RNA ligase and one single strand DNA ligase and (iii) a strand-displacing polymerase; (b) incubating the reaction mixture of step (a) under conditions that permit hybridization of the at least one ligation probe set to the target sequence present in the biological sample; (c) circularizing the probe set and amplifying the circularized probe by strand-displacement amplification; and (d) detecting and localizing target nucleic acid sequences (e.g. RNA sequences) in situ. In certain embodiments, the strand displacing DNA polymerase comprises Phi29 polymerase or Bst polymerase.

In certain embodiments, the hemi-bridge oligonucleotides and the ligation probe set are annealed simultaneously when contacting the sample. In certain embodiments, the hemi-bridge oligonucleotides comprise an overhanging 5′ end and a 3′ recessed end. In certain embodiments, the hemi-bridge oligonucleotides comprise a recessed 5′ end and a 3′ overhanging end.

In certain embodiments, the hemi-bridge oligonucleotides comprise blunt 5′ and 3′ ends. In certain embodiments, the target sequence is a ribonucleic acid (RNA) or a deoxyribonucleic acid (DNA) sequence. In certain embodiments, the biological sample is fixed (i.e. chemically crosslinked).

In certain embodiments, the fixed biological sample comprises fixed tissue, frozen-fixed tissue, formalin fixed paraffin embedded tissue, adherent fixed cells, suspension fixed cells or fixed cells.

In certain embodiments, one of ligases is a DNA ligase, including T4 DNA ligase or PBCV-1 DNA Ligase or Chlorella virus DNA Ligase. In certain embodiments, one of ligases is an RNA ligase, including T4 RNA ligase 2.

In certain embodiments, the polymerase is a strand displacing DNA polymerase. In certain embodiments, the strand displacing DNA polymerase comprises Phi29 polymerase or Bst polymerase.

In certain embodiments, the at least one ligase and the polymerase contact the biological sample simultaneously.

In certain embodiments, the at least one ligase contacts the biological sample as a first enzymatic step, followed by the polymerase as a second enzymatic step.

In certain embodiments, the method further comprises the step of identifying the location of the target RNA in the sample.

In certain embodiments, the method further comprises the step of quantifying the target RNA in the sample.

In certain embodiments, hybridization and the amplification are performed simultaneously.

In certain embodiments, hybridization and the amplification are performed sequentially.

In certain embodiments, the amplification step comprises rolling circle amplification.

In certain embodiments, the probe set comprises a barcode unique to the target RNA and wherein sequencing of the barcode detects the target RNA.

In certain embodiments, the sequencing comprises sequencing by synthesis or sequencing by ligation.

In certain embodiments, the target RNA is a viral RNA, a bacterial RNA, a fungal RNA, a nematode RNA, a human RNA, a non-human mammal RNA, a non-mammalian animal RNA or combinations thereof.

In certain aspects, a kit is provided for. In certain embodiments, a kit comprises a target-specific ligation probe set; and a hemi-bridge oligonucleotide.

In certain embodiments, the method further comprises at least one ligase, a strand-displacing polymerase or the combination thereof.

In certain embodiments, the ligase the ligase comprises T4 RNA Ligase 2 (Rnl2), a Chlorella virus DNA ligase (PBCV-1 DNA Ligase), a T4 DNA Ligase, and derivatives thereof.

In other embodiments, the kit further comprises a strand displacing DNA polymerase for amplifying by rolling circle amplification a circularized probe. The strand displacing DNA polymerase can comprise Phi29 polymerase or Bst polymerase.

### Definitions

It is understood that the present disclosure is not limited to the particular methods and components, etc., described herein, as these may vary. It is also to be understood that the terminology used herein is used for the purpose of describing particular embodiments only, and is not intended to limit the scope of the present disclosure. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this disclosure belongs. Specific methods, devices, and materials are described, although any methods and materials similar or equivalent to those described herein can be used in the practice or testing of the present disclosure.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present disclosure.

### Definitions

“Detect” refers to identifying the presence, absence, or amount of the nucleic acid (e.g., RNA) to be detected.

By “detectable label” is meant a composition that when linked to a molecule of interest renders the latter detectable, via, for example, spectroscopic, photochemical, biochemical, immunochemical, or chemical means. For example, useful labels may include radioactive isotopes, magnetic beads, metallic beads, colloidal particles, fluorescent dyes, electron-dense reagents, enzymes (for example, as commonly used in an ELISA), biotin, digoxigenin, mass-spectroscopically resolvable small molecules, or haptens.

By “fragment” is meant a portion of a nucleic acid molecule or polypeptide. This portion contains, or at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds. Unless noted otherwise, the term “annealing” is used synonymously with “hybridization” and the term “anneal” is used synonymously with “hybridize”.

By “marker” is meant any protein or polynucleotide having an alteration in expression level or activity that is associated with a disease or disorder. The term “biomarker” is used interchangeably with the term “marker.”

By “multi-partite” is meant having several or many parts or divisions.

By “multi-partite probe set” is meant a probe set having multiple parts or divisions. As an example, a multi-partite probe set of the present disclosure may comprise (i) a first multi-partite probe comprising a 5′ phosphorylated donor probe, at least one detection probe and a first bridge probe, wherein the 5′ phosphorylated donor probe specifically hybridizes to the target RNA and (ii) a second multi-partite probe comprising a 3′ acceptor probe, at least one detection probe and a second bridge probe, wherein the 3′ acceptor probe specifically hybridizes to the target RNA adjacent to the 5′ donor probe and the second bridge probe is 5′ phosphorylated. The terms “Lock'n'Roll probe sets”, “Lock'n'Roll probes”, “LnR probe sets” or “LnR probes” are all synonymous with or mean the same as “Multi-partite probe set”.

“Multi-partite probe set” is also meant a probe set having multiple parts or divisions. As an example, a multi-partite probe set of the present disclosure may comprise (i) a first multi-partite probe comprising a 5′ phosphorylated donor probe and at least one detection probe, wherein the 5′ phosphorylated donor probe specifically hybridizes to the target RNA and (ii) a second multi-partite probe comprising a 3′ acceptor probe and at least one detection probe, wherein the 3′ acceptor probe specifically hybridizes to the target RNA adjacent to the 5′ donor probe and the second bridge probe is 5′ phosphorylated. In this example of multi-partite probe set, either one of the donor or acceptor probe sets contains a binding site for an oligonucleotide primer that can be used for amplification of the circularized multi-partite probe set. The terms “Lock'n'Roll probe sets”, “Lock'n'Roll probes”, “LnR probe sets” or “LnR probes” are all synonymous with “Multi-partite probe set”.

“Laser capture microdissection” or “LCM” is a method for isolating specific cells from microscopic regions of tissues, cells or organisms. LCM is a method to procure subpopulations of tissue cells under direct microscopic visualization. LCM technology can harvest the cells of interest directly or can isolate specific cells by cutting away unwanted cells to give histologically pure enriched cell populations.

Ligation in situ Hybridization or “LISH” is a methodology disclosed in US 2018/0208967 (U.S. application Ser. No. 15/747,245) incorporate herein by reference to Larman et al. which includes multiplexed measurement of gene expression, suited for analysis of fixed tissue specimens. One embodiment of LISH utilizes the T4 RNA Ligase 2 (Rnl2) and chimeric DNA-RNA hybridization probes, which when annealed, then become ligated, where they serve as faithful proxies to a respective target's expression level.

LISH-Lock'n'Roll as referred to herein including LISH-Lock'n'Roll methods have been disclosed in US 2023/0039899 (U.S. application Ser. No. 17/790,293) to Larman et al. incorporated herein by reference, which in certain aspects can include methods for detecting an immobilized target ribonucleic acid (RNA) comprising the steps of: a) contacting a biological sample comprising the target RNA in a reaction mixture with at least one probe set comprising (i) a first multi-partite probe comprising a 5′ phosphorylated donor probe and a first bridge probe, wherein the 5′ phosphorylated donor probe specifically hybridizes to the target RNA; and (ii) a second multi-partite probe comprising a 3′ acceptor probe and a second bridge probe, wherein the 3′ acceptor probe specifically hybridizes to the target RNA adjacent to the 5′ donor probe and the second bridge probe is 5′ phosphorylated; b) incubating the reaction mixture of step (a) under conditions that permit hybridization of the at least one probe set to the target RNA present in the biological sample; c) washing away unbound probe sets; d) d, ligating the 5′ phosphorylated donor probe and the 3′ acceptor probe; e) contacting the reaction mixture with at least one bridge primer that specifically hybridizes to the first bridge probe and the second bridge probe, wherein the first bridge probe and the second bridge probe anneal to the bridge primer adjacent to each other; f) ligating the first bridge probe and the second bridge probe thereby forming a circularized probe that is hybridized to the target RNA; g) amplifying the circularized probe by rolling circle amplification; and h) detecting the target RNA which detecting may include sequencing of rolling circle amplification products.

By “pathogen” is meant anything that can produce a disease including a bacterium, virus, fungi or other microorganism, as examples.

By “infection” is meant the invasion of an organism's body by disease-causing agents, their multiplication and the reaction of the host to these organisms and the toxins they produce. The infection may be caused by any microbes/microorganisms, including for example, bacteria, fungi, and viruses. Microorganisms can include all bacterial, Archaean, and the protozoan species. This group also contains some species of fungi, algae, and certain animals. In some embodiments, viruses may be also classified as microorganisms.

By “reduces” is meant a negative alteration of at least 10%, 25%, 50%, 75%, or 100%.

By “reference” is meant a standard or control conditions such as a sample (human cells) or a subject that is a free, or substantially free, of an agent such as a pathogen.

By “reference sequence” is meant a defined sequence used as a basis for sequence comparison. A reference sequence may be a subset of or the entirety of a specified sequence; for example, a segment of a full-length cDNA, RNA, or gene sequence, or the complete cDNA, RNA, or gene sequence. For polypeptides, the length of the reference polypeptide sequence will generally be at least about 16 amino acids, or at least about 20 amino acids, more or at least about 25 amino acids, and even more or about 35 amino acids, about 50 amino acids, or about 100 amino acids. For nucleic acids, the length of the reference nucleic acid sequence will generally be at least about 40 nucleotides, or at least about 60 nucleotides, more or at least about 75 nucleotides, or about 100 nucleotides or about 300 nucleotides or any integer thereabout or there between.

By “sensitivity” is meant a percentage of subjects correctly identified as having a particular disease or condition, or pathogen.

By “specificity” is meant a percentage of subjects correctly identified as NOT having a particular disease or condition, or pathogen, i.e., normal or healthy subjects.

By “specifically binds” is meant a multi-partite probe set that recognizes and binds a nucleotide sequence of the disclosure, but which does not substantially recognize and bind other molecules in a sample, for example, a biological sample, which naturally includes nucleotide sequences unrelated to the disclosure. In some embodiments, a 5′ phosphorylated donor probe and a 3′ acceptor probe specifically hybridize or bind to a target RNA. In other embodiments, a genotyping probe specifically binds a target nucleic acid having a particular single nucleotide polymorphism (SNP) but does not specifically bind a nucleic acid having an alternative SNP.

By “subject” is meant any individual or patient to which the method described herein is applied. Generally, the subject is human, although as will be appreciated by those in the art, the subject may be an animal (e.g., pet, agricultural animal, wild animal, etc.), disease vector (e.g., mosquitoes, sandflies, triatomine bugs, blackflies, ticks, tsetse flies, mites, snails, lice, etc.), or an environmental sample (e.g., sewage, food products, etc.). Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Nucleic acid molecules useful in the methods of the disclosure need not be 100% identical with an endogenous nucleic acid sequence but will typically exhibit substantial identity. Polynucleotides having “substantial identity” to an endogenous sequence are typically capable of hybridizing with a target molecule. By “hybridize” is meant pair to form a double-stranded molecule between complementary polynucleotide sequences, or portions thereof, under various conditions of stringency. See, e.g., Wahl, G. M. and S. L. Berger (1987) Methods Enzymol. 152:399; Kimmel, A. R. (1987) Methods Enzymol. 152:507. For example, stringent salt concentration will ordinarily be less than about 750 mM NaCl and 75 mM trisodium citrate, or less than about 500 mM NaCl and 50 mM trisodium citrate, and more or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more or at least about 50% formamide. Stringent temperature conditions will ordinarily include temperatures of at least about 30° C., more or of at least about 37° C., and most or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will or be less than about 30 mM NaCl and 3 mM trisodium citrate, and most or less than about 15 mM NaCl and 1.5 mM trisodium citrate. Stringent temperature conditions for the wash steps will ordinarily include a temperature of at least about 25° C., more or of at least about 42° C., and sometimes above 50° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

“Sequencing” or any grammatical equivalent as used herein may refer to a method used to sequence the amplified target nucleic acid proxy. The sequencing technique may include, for example, Next Generation Sequencing (NGS), Deep Sequencing, mass spectrometry-based sequence or length analysis, or DNA fragment sequence or length analysis by gel electrophoresis or capillary electrophoresis. Compatible sequencing techniques may be used including single-molecule real-time sequencing (Pacific Biosciences), Ion semiconductor (Ion Torrent sequencing), pyrosequencing (454), sequencing by synthesis (Illumina), sequencing by ligation (SOLiD sequencing), chain termination (Sanger sequencing), Nanopore DNA sequencing (Oxford Nanosciences Technologies), Helicos single molecule sequencing (Helicos Inc.), sequencing with mass spectrometry, DNA nanoball sequencing, sequencing by hybridization, and tunneling currents DNA sequencing.

By “NGS” is meant Next Generation Sequencing. NGS platforms perform massively parallel sequencing, during which millions of fragments of DNA from a single sample are sequenced in unison. Massively parallel sequencing technology facilitates high-throughput sequencing, which allows an entire genome to be sequenced in less than one day. The creation of NGS platforms has made sequencing accessible to more labs, rapidly increasing the amount of research and clinical diagnostics being performed with nucleic acid sequencing.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60%, more or 80% or 85%, and more or 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

“Primer set” means a set of oligonucleotides that may be used, for example, in a 30 polymerase chain reaction (PCR). A primer set comprises at least 2, 4, 6, 8, 10, 12, 14, 16, 18, 20, 30, 40, 50, 60, 80, 100, 200, 250, 300, 400, 500, 600, or more primers.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges”that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, the term “sub-probe” may refer to any of the two or more probes that bind the contiguous target sequence without leaving any unbound intervening nucleotides. In some embodiments, the multi-partite probe described herein may include at least two “sub-probes.” In another embodiment, each of the target binding sites of the at least two sub-probes of the plurality of multi-partite probes may be about 10-50 nucleotides in length. Once the probes are ligated, the ligated multi-partite probe (alternatively, the “ligated sub-probe”) may be released from the RNA. In some embodiments, the sub-probe may contain appended primer binding site (e.g., adapters) to facilitate subsequent amplification of the target nucleic acid proxy. In other embodiments, at least one of the two or more sub-probes may be referred to as “acceptor sub-probes” that have a 3′-termination of at least two RNAbases.

As used herein, “appended primer binding” sites may refer to binding sites within the multi-partite probe or sub-probes described herein that facilitate amplification of the target nucleic acid proxy. “Appended primer binding sites” may also be referred to as “adapters.”

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D are schematics showing various Bridge primer formats. FIG. 1A: The Bridge Standard format, requires sequential addition. The Bridge primer must be added after the LnR ligation probes or else will drive a high degree of non-specific and random LnR ligation probe pairing and background noise. FIG. 1B: The Hemi-bridge format Bridge Overhang enables simultaneous LnR ligation probe and Hemi-bridge primer annealing, and features “sticky” ends to potentially enhance circularization. FIG. 1C: The Hemi-bridge format Bridge Recessed enables simultaneous LnR ligation probe and Hemi-bridge primer annealing, and features “sticky” ends to potentially enhance circularization. FIG. 1D: The Hemi-bridge format Bridge Blunt enables simultaneous LnR ligation probe and Hemi-bridge primer annealing. This format does not involve “sticky” ends to potentially enhance circularization.

FIGS. 2A, 2B are schematics that demonstrate that hemi-bridge formats enable a more streamlined workflow. FIG. 2A: Workflows involving the Bridge Standard format require separation of LnR ligation probe annealing and Hemi-bridge annealing (Steps I and II). FIG. 2B: Workflows involving the Hemi-bridge format (Bridge Blunt shown here) can combine LnR ligation probe and Hemi-bridge annealing steps (Step 1). In this streamlined workflow, all three enzymes are added for ligation and phi29 rolling circle amplification (RCA).

FIGS. 3A, 3B are a series of photographs of assayed cells and graphs demonstrating the performance of Hemi-bridge versus Bridge Standard formats. FIG. 3A: The assay performance of the 3 Hemi-bridge formats (Bridge Recessed, Bridge Overhang, and Bridge Blunt) were all compared against the Bridge Standard format for detection of GAPDH, RPL19 and p-actin in A59 cells. LnR ligation probes were annealed and then the Hemi-bridge or Bridge Standard primers were applied sequentially. Image shows RCA spots decoded using a three-color codebook. FIG. 3B: Quantification of the spots/cell shown in A for GAPDH, RPL19 and f3-actin. Mismatched probe sets were also quantified (bar plot on right).

FIGS. 4A, 4B are a series of photographs of assayed cells and graphs demonstrating the performance of Hemi-bridge in combined hybridization versus sequential hybridization workflows. FIG. 4A: The assay performance of the Hemi-bridge format (Bridge Blunt primer in this example) was assessed in a sequential hybridization workflow with triple enzyme incubation at 37° C. or 30° C., versus a combined hybridization workflow (LnR ligation probes plus Bridge Blunt primer) with triple enzyme incubation at 30° C. As in FIGS. 2A, 2B, detection of GAPDH, RPL19 and β-actin in A59 cells served as the model system. Image shows RCA spots decoded using a three-color codebook. FIG. 4B: Quantification of the spots/cell shown in A for GAPDH, RPL19 and β-actin. Mismatched probe sets were also quantified (bar plot on right).

FIGS. 5A, 5B are schematics that demonstrate probe ligation formats involving use of a single stranded DNA ligase. In FIG. 5A, the oligonucleotide primer that primes Phi29 rolling circle amplification does not bridge the pair of ligation probes. Rather, the primer is annealed to just one of the two ligation probes. It is shown that the primer is binding to the 3P acceptor probe in this drawing, but it can be annealed to either the donor or acceptor ligation probes, putting the ssDNA ligation junction proximal to the 5′ or 3′ end of the primer, respectively. The final in situ amplification products will be essentially or effectively identical. FIG. 5B is a schematic that shows the same approach as FIG. 5A, but which involves placement of the ssDNA ligase junction between two readout probe binding sites. In this embodiment, the ssDNA ligation joins together two sequences (in this example RP1 and RP2), which may create a unique sequence comprising the combination of RP1 and RP2 (e.g. “RP1-RP2”). Such an approach may be of particular utility for in situ sequencing of the combined sequence (e.g. RP1-RP2), or for in situ hybridization-based detection with a labeled probe specific for the combined sequence (e.g. RP1-RP2) or a subsequence thereof. FIG. 5C is a schematic that demonstrates an assay workflow involving the ssDNA ligation approach. The annealing step can combine the LnR ligation probes and the oligonucleotide primer (Step I). In this streamlined workflow, all three enzymes are added for ligation and phi29 rolling circle amplification (RCA).

FIGS. 6A-6C show results of Example 2 which follows. In FIG. 6A, two Ligation In Situ Hybridization Lock'n'Roll (LISH-LnR) probe sets are depicted targeting two distinct mRNA target sequences in fixed tissue. FIG. 6B is a schematic that includes photographs of A549 cells cultured on a microscope slide and then fixed and then prepared for LISH-LnR analysis. FIG. 6C shows quantification of the mass spectrometry tags corresponding to the images of FIG. 6B.

### DETAILED DESCRIPTION

In one aspect, we now provide a multiplexed probe ligation method, which can be used to track RNA abundance and position with minimal cost, uses common laboratory instrumentation and a requires a relatively simple workflow. The high level of signal amplification with Lock'n'Roll using a single probe set provides several unique advantages over smFISH-based and in situ sequencing methods. One, robust amplification of the Lock'n'Roll probes, makes it possible to detect any RNA sequence in situ with a single probe set instead the >40 probes/target required for methods such as MERFISH. Second, because Lock'n'Roll uses a single target-identifying probe set, greater discrimination of RNA sequences of interest is possible based on the presence or absence of SNP's, mutations, novel splicing isoforms, and fusions. Third, Lock'n'Roll can be accomplished in one or two days unlike other methods (e.g., FISSEQ), which may require several days to weeks to complete. Lock'n'Roll's commercial advantage is its accuracy, simplicity and cost, features that will certainly foster its widespread adoption among academic and clinical labs beyond other in situ transcriptome platforms.

In one aspect, methods are provided to perform a more streamlined Lock'n'Roll assay, which involves the use of a Hemi-bridge primer format. Hemi-bridge formats enable the simultaneous hybridization of LnR ligation probes and bridge primer. This single oligonucleotide hybridization step can be followed by a combined ligase (e.g. Rnl2 and T4 DNA ligase and or a single stranded DNA ligase) and Phi29 polymerase step. The resulting streamlined protocol minimizes hands on time, without sacrificing assay performance. Streamlined Lock'n'Roll assays can be used for low to highly multiplexed spatial RNA analyses. LnR ligation probes can be designed to distinguish even closely related RNA molecules and the assay can likely be combined with other complementary-omics techniques including MERFISH and antibody staining assays when desired.

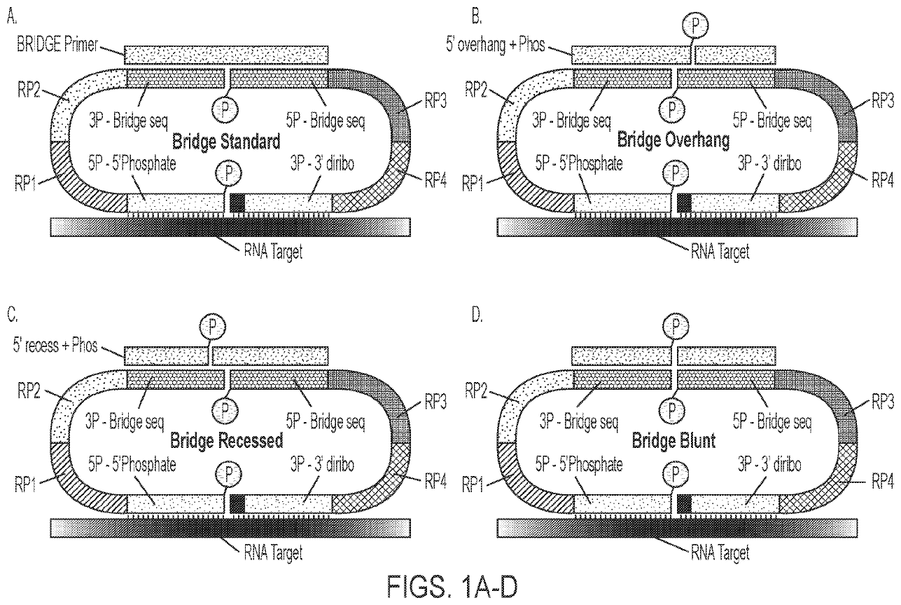

As referred to herein, a hemi-bridge oligonucleotide (as opposed to a full bridge oligonucleotide which is intact and spans the two probe) does not bring the two probes together unless the two probes are annealed to a target sequence. FIGS. 1A-ID show various exemplary Bridge primer formats. The Bridge Standard format (FIG. 1A) requires sequential addition. The Bridge primer must be added after the LnR ligation probes or else will drive a high degree of non-specific and random LnR ligation probe pairing and background noise. The Hemi-bridge format Bridge Overhang (FIG. 1B) enables simultaneous LnR ligation probe and Hemi-bridge primer annealing, and features “sticky” ends to potentially enhance circularization. The Hemi-bridge format Bridge Recessed enables simultaneous LnR ligation probe and Hemi-bridge primer annealing, and features “sticky” ends to potentially enhance circularization (FIG. 1C). The Hemi-bridge format Bridge Blunt enables simultaneous LnR ligation probe and Hemi-bridge primer annealing (FIG. 1D). In certain embodiments the hemi-bridge oligonucleotides comprise one or more bridge nucleic acids. Examples of bridge oligonucleotides can be found in Soler-Bistué, A. et al., Bridged Nucleic Acids Reloaded. Molecules 2019, 24, 2297. doi.org/10.3390/molecules24122297.

### LISH-Lock'n'Roll

Hybridization of probes in a sample, followed by in situ ligation (“LISH”), locks specifically circularized probe set around an RNA target sequence. Rolling circle amplification (“LISH-Lock'n'Roll”), followed by fluorescently labeled detector probe hybridization, enables simultaneous in situ quantification and localization of RNA sequences with subcellular precision. This technology is a time and cost-effective alternative to other in situ RNA analysis methods.

In prior work, the inventors have described the utility of T4 RNA Ligase 2 (Rnl2), an enzyme that performs RNA-templated ligations of DNA probes with very high efficiency, when the two 3′ bases of the acceptor probe are composed of ribonucleotides^{17 18}. This ligation chemistry enables the multiplexed quantification of RNA in a high throughput assay referred to as RNA-mediated oligonucleotide Annealing, Selection, and Ligation with sequencing (‘RASL-seq’). The present inventors have also applied this ligation chemistry to the analysis of RNA sequences in formalin fixed tissue specimens in an assay called Ligation In Situ Hybridization sequencing (LISH-seq). Here, the inventors present LISH-Lock'n'Roll, which is a novel, yet related approach for multiplexed quantification and localization of RNA sequences in a fixed biological specimen. Target RNA sequences are preferably greater than 40 nucleotides long and may be associated with host or infectious disease-specific RNA transcripts.

The LISH-Lock'n'Roll probe set is suitably composed of a 3′ acceptor probe and a 5′ donor probe (LnR probe set). The 3′ terminus of the acceptor probe set is composed of two ribonucleic acid bases at the 3′ end, which foster high efficiency ligation by the T4 RNA ligase 2, Rnl2. The 5′ donor probe is phosphorylated at the 5′ end. The probe set's targeting sequences are designed to anneal adjacent to one another on the RNA target. The targeting sequences can be roughly 20 nucleotides, but may also be substantially longer or shorter. Only when the LnR probes are annealed adjacent to one another on a target sequence can they be ligated together via Rnl2. This requirement that the ligation probes anneal to adjacent sequences provides a high level of assay specificity. Additionally, each acceptor and donor probe feature one or two 30-nucleotide detector sequences and a 17-nucleotide bridge sequence. In step 1 and 2, the LnR acceptor and donor probes anneal to the target RNA sequence, followed by ligation with Rnl2.

Excess probes are then washed away. After adjacent donor and acceptor probe have become ligated, will the two probe halves present the complete 34-nucleotide bridge sequence (17 nt from each probe), which is subsequently hybridized by the bridge primer (step 3) and ligated by T4 DNA ligase (step 4). At this stage, the probe sets have been ligated at both ends, completing a circle, such that due to the twist of the double helix, locks it into place around the target RNA. Phi29 polymerase is then added to the tissue, enabling rolling circle amplification (RCA) to take place in situ, as it is primed by the annealed bridge primer that was used for circularization (step 5). The RCA product is in essence a “nanoball” of single stranded DNA containing many copies of the detector sequences. Due to the extensive crosslinking of the surrounding tissue, the nanoball remains trapped in a position that approximates the position of the templating RNA molecule. Following completion of RCA, fluorescently labeled oligos (detector probes) are annealed to the complementary detector sequences, of which there are now many spatially localized copies (step 6). The tissue is now ready to be processed for imaging.

Probe sets are typically designed to have 1 to 4 unique detector sequences. Target multiplexing is the use of probe sets that target different mRNA transcripts using distinct detector sequences. Multiplexing based on color barcoding, wherein a probe set has two or more distinct detector sequences for simultaneous or sequential binding of two or more distinctly colored detector probes. Color barcoding enables a greater level of combinatorial multiplexing, as well as the opportunity for encoding error-correcting color combinations. Target multiplexing, combined with color barcoding, enables simultaneous spatial quantification of many different mRNA transcripts. As an example, a panel of LnR probe sets with two different detector sequences per probe set, and five uniquely colored detector probes, can be used to simultaneously measure more than 15 targets during a single cycle of imaging. Additional rounds of probe removal and detector probe hybridization can exponentially amplify the level of multiplexing achievable.

To determine the efficiency and specificity of LiSH-Lock'n'Roll target multiplexing, the present inventors designed probe sets targeting GAPDH and β-actin for use in fixed HeLa cells as a model tissue. Each probe set could be bound by only one of two spectrally distinct fluorophore-labeled detector probes, detector-1 (GAPDH, Alexa-488 labeled oligo) and detector-2 (β-actin, Alexa-647 labeled oligo). The zoomed images (white box in main image) showing the individual detector probes when overlaid, revealed no spatial overlap of the individual detector probes. Phi29-dependent amplification of the locked circle was >1,000-fold higher than unamplified samples when the products were measured by qPCR using primers specific to the individual targeting sequences. Average spots per cell and average spot diameter were calculated using imageJ software. There were 150+/−50 spots/cell for b-actin and 270+/−70 spots/cell for GAPDH with both probe sets producing spot sizes of 50-500 nM in diameter. No spots were detectable when Phi29 was omitted revealing the high degree of specificity achievable with the LISH-Lock'n'Roll method. To determine the efficiency and specificity of LISH-Lock'n'Roll color multiplexing, the present inventors designed a single probe sets targeting R-actin that had two distinct detector sequences, which were also tested in fixed HeLa cells. The RCA product arising from this single probe set was equivalently bound by both of the spectrally distinct fluorophore-labeled detector probes, detector-1 (Alexa-488 labeled) and detector-2 (Alexa-647 labeled). As expected, the zoomed images (white box in main image) revealed complete overlap of the two detector probes. In this experiment, Phi29-dependent amplification of the locked circle was >600-fold higher than the unamplified samples when products were measured by qPCR using primers specific to the individual targeting sequences. As an example, a panel of LnR probe sets with four different detector sequences per probe set (two per probe), and five uniquely colored detector probes, can be used to simultaneously measure 30 targets during a single cycle of imaging. Additional rounds of probe removal and detector probe hybridization can exponentially amplify the level of multiplexing achievable.

Lock'n'Roll with a Standard Bride

The Lock'n'Roll ligation probe set is composed of a 3′ acceptor probe and a 5′ donor probe (LnR probe set). In one embodiment, the 3′ terminus of the acceptor probe set is composed of two ribonucleic acid bases at the 3′ end, which foster high-efficiency target RNA templated ligation by the T4 RNA ligase 2, Rnl2. The 5′ donor probe is phosphorylated at the 5′ end. The probe set's targeting sequences are designed to anneal adjacent to one another on the RNA target (FIG. 1A). The targeting sequences can be roughly 20 nucleotides but may also be substantially longer or shorter. Only when the LnR probes are annealed adjacent to one another on a target RNA sequence can they be ligated together via Rnl2. This requirement that the ligation probes anneal to adjacent sequences provides a high level of assay specificity. Additionally, each acceptor probe and donor probe contain one or two or 15-nucleotide readout probe binding sites and a 17-nucleotide sequence that will bind the “standard” bridge primer (FIG. 1A). The readout probe binding sites and the bridge primer binding site can be significantly longer or shorter than mentioned.

The previously standard Lock'n'Roll workflow begins with the LnR acceptor and donor probes annealing to the target RNA sequence, followed by ligation with Rnl2. Excess LnR ligation probes are then washed away. Only when adjacent donor and acceptor probe have become ligated, do the two probe halves present the complete 34-nucleotide sequence (17 nt from each probe) for binding of the Standard bridge primer. The Standard bridge primer enables the circularization of the ligation probe set by the action of T4 DNA ligase. Due to the twist of the double helix, this ligation event locks the LnR probe set into place around the target RNA molecule. Phi29 polymerase is then added to initiate rolling circle amplification (RCA), which is primed by the annealed bridge primer.

The RCA product is in essence a ‘nanoball’ of single stranded DNA containing many copies of detector probe binding sequences. Due to the extensive crosslinking of the surrounding tissue, the nanoball remains trapped in a position that approximates the position of the templating RNA molecule. Following completion of RCA, fluorescently labeled oligos (detector probes) are annealed to the complementary detector sequences, of which there are now many spatially localized copies. One or many rounds of hybridization and imaging can be performed to determine the location of the target RNA molecules. Probe sets can be designed to carry 1 to 4 or more unique detector probe binding sequences. Several rounds of hybridization and imaging can exponentially increase the level of multiplexing achievable. ‘Codebooks’ link the pattern of overlapping fluorescent signals to the identity of the ligation probe pair that initiated each RCA nanoball or “rolony”. Color barcoding enables a greater level of combinatorial multiplexing, as well as the opportunity for encoding error-correcting color combinations.

Lock'n'Roll with a Hemi-Bridge Design

Hybridization of probes in a sample, followed by in situ ligation, locks specifically circularized probe set around an RNA target sequence. Rolling circle amplification (“Lock'n'Roll”), followed by fluorescently labeled detector probe hybridization, enables simultaneous in situ quantification and localization of RNA sequences with subcellular precision. This technology is more accurate, and a time and cost-effective alternative to other in situ RNA analysis methods. These benefits of the Lock'n'Roll methodology will foster its widespread adoption.

The inventor(s) prior work, described the utility of T4 RNA Ligase 2 (Rnl2), an enzyme that performs RNA-templated ligations of DNA probes with very high efficiency, when the two 3′ bases of the acceptor probe are composed of ribonucleotides^{17,18}. This ligation chemistry enables the multiplexed quantification of RNA in a high throughput assay referred to as RNA-mediated oligonucleotide Annealing, Selection, and Ligation with sequencing (“RASL-seq”). We have also applied this ligation chemistry to the analysis of RNA sequences in formalin fixed tissue specimens in an assay called Ligation In Situ Hybridization sequencing (LISH-seq). Here we present Lock'n'Roll, which is a novel, yet related approach for multiplexed quantification and localization of RNA sequences in a fixed biological specimen. Target RNA sequences are or greater than 40 nucleotides long and may be associated with host or infectious disease-specific transcripts.

FIGS. 1A-1D disclose three new ways of implementing the Lock'n'Roll assay using a more streamlined workflow. The previously disclosed bridge primer design is referred to as “Bridge Standard” (FIG. 1A). In the present disclosure, we describe the use of Hemi-bridge design, which can be embodied in three different ways. The Hemi-bridge can take the form of an overhanging 5′ end and a 3′ recessed end (“Bridge Overhang”, FIG. 1B). Alternatively, the Hemi-bridge can take the form of a recessed 5′ end and a 3′ overhanging end (“Bridge Recessed”, FIG. 1C). Finally, the Hemi-bridge can take the form of blunt 5′ and 3′ ends (“Bridge Blunt”, FIG. 1D). To determine the efficiency and specificity of Lock'n'Roll assay performance, we used probe sets targeting GAPDH, RPL19 and f-actin in A59 cells as a surrogate tissue.

The present disclosure can be used to detect multiple target nucleic acids. In such multiplex embodiments, the at least one probe set is configured for multiplex detection of 1-30,000 distinct target nucleic acids. In other embodiments, the at least one probe set comprises a range of combined 1-20,000, 10-10,000, 20-5000, or 50-1000 probe sets. In particular embodiments, the compositions are configured to detect the presence or absence of SNPs, mutations, novel splicing isoforms, fusions and the like.

In other embodiments, more than one probe set can be designed to bind different locations/regions of the same nucleic acid. The number of rolling circle amplification (RCA) products formed per target nucleic acid can be decreased by adding unligatable probe sequences to the biological sample.

In certain embodiments, the probes range in size from 30-1000 nucleotides. In other embodiments, probes of the present disclosure may range in size from about 30 to about 1000 nucleotides, from about 25 to about 9000 nucleotides, about 30 to about 8000 nucleotides, about 25 to about 5000 nucleotides, about 40 to about 2000 nucleotides, about 50 to about 1000, or about 30 to about 200 nucleotides.

The target RNA can be a viral RNA, a bacterial RNA, a fungal RNA, a nematode RNA, a human RNA, a non-human mammal RNA, a non-mammalian animal RNA or combinations thereof.

In particular embodiments, the present disclosure is used to detect an immobilized target RNA. In a specific embodiment, the RNA is immobilized as part of a fixed biological sample comprising the target RNA. The fixed biological sample can comprise fixed tissue, frozen-fixed tissue, formalin fixed paraffin embedded tissue, adherent fixed cells, suspension fixed cells or fixed cells.

In some methods of the present disclosure, the fixed biological sample comprises cells and the location of the rolling circle amplification products in the sample is used to infer the type or phenotype of a cell or cells. In some methods of the present disclosure, the fixed biological sample is tissue processed into sections having a thickness of 1-1000, 10-900, 20-800, 30-500, or 40-200 microns.

In particular embodiments, RCA products can be immobilized within the sample by crosslinking the RCA product to the biological sample. Crosslinking may occur by applying a reagent to the RCA products, wherein the reagent is paraformaldehyde, formaldehyde, formalin, glutaraldehyde, osmium tetroxide, potassium dichromate, chromic acid, and potassium permanganate, and Hepes-glutamic acid buffer-mediated organic solvent fixative, or a combination thereof.

Bridge primers used in the present disclosure may include a reactive moiety and RCA products may be immobilized within the sample by the reactive moiety on the bridging primer.

In alternative embodiments, the target RNA is immobilized by capture. A labeled target RNA capture probe can be used including, but not limited to, biotin, diogexin, acrydite, haloalkane, or click chemistry. A capture element can include avidin, streptavidin, neutravidin, anti-digoxin antibodies, click chemistry, halo protein, or a combination thereof. A solid support can be used to capture target RNA, and can include magnetic material, polystyrene, agarose, silica, lateral flow strip, microfluidic chambers, or a combination thereof can be used in the immobilization process.

In particular embodiments, the ligating step is performed using a ligase selected from the group consisting of T4 RNA Ligase 2 (Rnl2), a Chlorella virus DNA ligase (PBCV-1 DNA Ligase), a T4 DNA Ligase, derivatives thereof, and combinations thereof.

In particular embodiments, the detecting step comprises sequencing or hybridization.

In certain embodiments, detecting step (h) comprises sequencing of rolling circle amplification products. All or a portion of the rolling circle amplification products can be sequenced. In a specific embodiment, the probe set comprises a barcode unique to the target RNA and wherein sequencing of the barcode detects the target RNA. In a more specific embodiment, the sequencing comprises sequencing by synthesis or sequencing by ligation. In an even more specific embodiment, the method is performed n situ on a fixed sample. In an alternative embodiment, the sequencing comprises sequencing by synthesis, and the synthesized sequence creates a unique color barcode that detects the target RNA.

In other embodiments, the detecting step comprises sequencing the ligated sequence. In yet another embodiment, detecting step comprises contacting the reaction mixture with a detectably labeled detector probe that specifically hybridizes the ligated sequence.

In a specific embodiment, the method can further comprise the step of identifying the location of the target RNA in the sample. In another embodiment, the method further comprises the step of quantifying the target RNA in the sample.

Examples of detector probes used in the present disclosure include fluorescently labeled nucleic acid sequences in the range of 10 to 100 nucleotides bound to a detection element. The detector probes are fluorescently labeled with a fluorescent probe such as a fluorophore, fluorescent protein, quantum dot, biotin, digoxin, heavy metal or small molecule mass tag, surface-enhanced Raman scattering tag, or peroxidase enzyme.

In further embodiments, multiple rounds of hybridization and stripping, quenching or bleaching of detection probes can be performed. In a specific embodiment, the detection probes hybridize to detection sequences present in the probe set. In another specific embodiment, the detection probes hybridize to barcodes present in the probe set. In a non-limiting embodiment, RCA products are prepared on, for example, 1000 targets, creating nanoballs of DNA in the tissue. Ten rounds of hybridization with fluorescent probes are conducted. The probes are stripped off or otherwise destroyed and the process is repeated until all of the targets are detected. If the probe set comprises multiple detection probes, then different combinations of detection probes can be used to identify the different target nucleic acids. In particular embodiments, amplification step is performed using a strand displacing DNA polymerase. In specific embodiments, the strand displacing DNA polymerase comprises Phi29 polymerase or Bst polymerase.

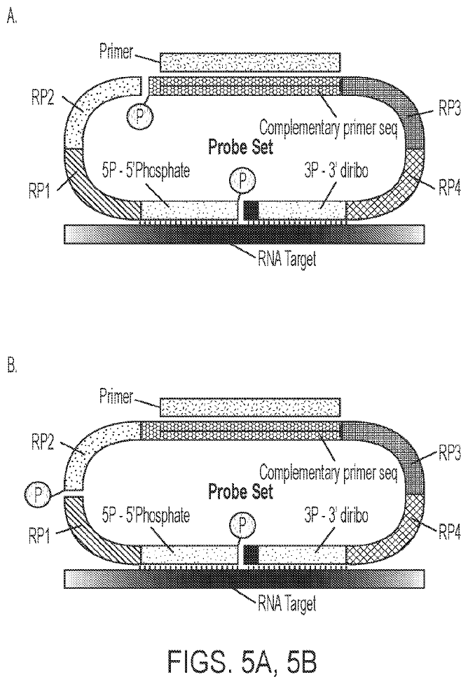

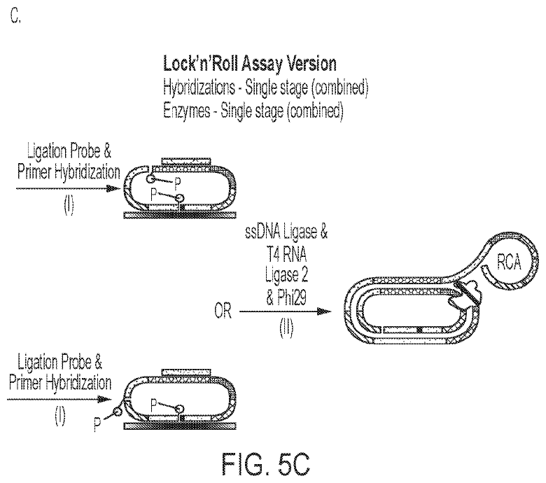

FIGS. 5A, 5B are schematics that demonstrate exemplary probe ligation formats involving use of a single stranded DNA ligase.

In FIG. 5A, the oligonucleotide primer that primes Phi29 rolling circle amplification does not bridge the pair of ligation probes. Rather, the primer is annealed to just one of the two ligation probes. It is shown that the primer is binding to the 3P acceptor probe in this drawing, but it can be annealed to either the donor or acceptor ligation probes, putting the ssDNA ligation junction proximal to the 5′ or 3′ end of the primer, respectively. The final in situ amplification products will be essentially or effectively identical.

FIG. 5B is a schematic that shows the same approach as FIG. 5A, but which involves placement of the ssDNA ligase junction between two readout probe binding sites. In this embodiment, the ssDNA ligation joins together two sequences (in this example RP1 and RP2), which may create a unique sequence comprising the combination of RP1 and RP2 (e.g. “RP1-RP2”). Such an approach may be of particular utility for in situ sequencing of the combined sequence (e.g. RP1-RP2), or for in situ hybridization-based detection with a labeled probe specific for the combined sequence (e.g. RP1-RP2) or a subsequence thereof. FIG. 5C is a schematic that demonstrates an assay workflow involving the ssDNA ligation approach. The annealing step can combine the LnR ligation probes and the oligonucleotide primer (Step 1). In this streamlined workflow, all three enzymes are added for ligation and phi29 rolling circle amplification (RCA).

### Kits

Any of the compositions described herein may be comprised in a kit. In a non limiting example a kit comprises a target-specific ligation probe set; and a hemi-bridge oligonucleotide. In certain embodiments, the kit further comprises at least one ligase, a strand-displacing polymerase or the combination thereof.

The ligase comprises T4 RNA Ligase 2 (Rnl2), a Chlorella virus DNA ligase (PBCV-1 DNA Ligase), a T4 DNA Ligase, and derivatives thereof. The kit can further comprise a ligase for ligating the first bridge probe and the second bridge probe to form a circularized probe that is hybridized to the target nucleic acid.

In other embodiments, the kit further comprises a strand displacing DNA polymerase for amplifying by rolling circle amplification a circularized probe formed by ligating the first bridge probe and the second bridge probe and hybridized to the target nucleic acid. The strand displacing DNA polymerase can comprise Phi29 polymerase or Bst polymerase.

The kits may comprise a suitably sized aliquot of any of the compositions comprised herein and, in some cases, one or more additional agents such as buffers, for example. The component(s) of the kits may be packaged either in aqueous media or in lyophilized form.

The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing any of the compositions described herein and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means within the kit.

All documents (including patent applications) disclosed herein are fully incorporated herein by reference in their entirety.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

### Example 1: Multiplexed and Spatially Resolved Analysis of RNA with a Streamlined Workflow

It is demonstrated herein, that hybridization of probes in a sample, followed by in situ ligation, locks specifically circularized probe sets around an RNA target sequence. Rolling circle amplification (“Lock'n'Roll”), followed by fluorescently labeled detector probe hybridization, enabled simultaneous in situ quantification and localization of RNA sequences with subcellular precision.

To determine the efficiency and specificity of Lock'n'Roll assay performance, probe sets were used that targeted GAPDII, RPL19 and β-actin in A59 cells as a surrogate tissue.

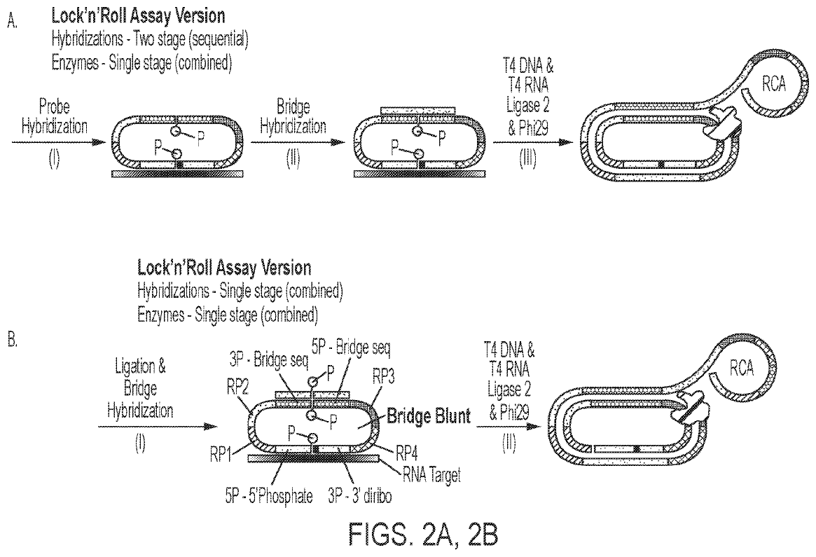

The key workflow advantage of the Hemi-bridge system versus the Standard bridge system is that the Hemi-bridge oligos can be pre-annealed on the ligation probes or annealed simultaneously with the ligation probes inside the tissue, thereby saving a step compared to the previous Lock'n'Roll invention. This workflow is simplified because the Standard bridge design requires separated steps of first annealing the ligation probes, then washing, and then incubation with the Standard bridge primer (steps I and II of FIG. 2A). These separate steps are necessary because if the Standard bridge primer were to be introduced simultaneously with the ligation probes, the bridge primer would nonspecifically and randomly bring together the ligation probes and likely lead to a very high level of background noise. FIG. 2B shows the new and simplified Hemi-bridge workflow, which can involve simultaneous annealing of the ligation probes and a Hemi-bridge design, which does not increase assay background noise. Bridge Blunt is shown as an example in FIG. 2B. In FIG. 2A-B, the workflow indicates both the Rnl2 and T4 DNA ligases, along with Phi29, being used simultaneously. For some experiments disclosed herein, the three enzymes are used simultaneously, which represents the most streamlined version of the workflow. Occasionally where indicated, however, the two ligases (Rnl2 and T4 DNA ligase) are used in a first enzymatic step, and the Phi29 polymerase is used in a second enzymatic step.

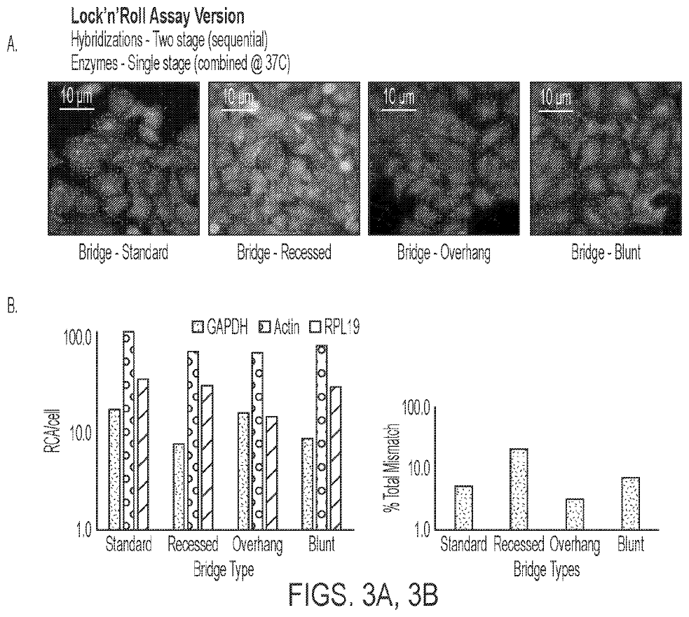

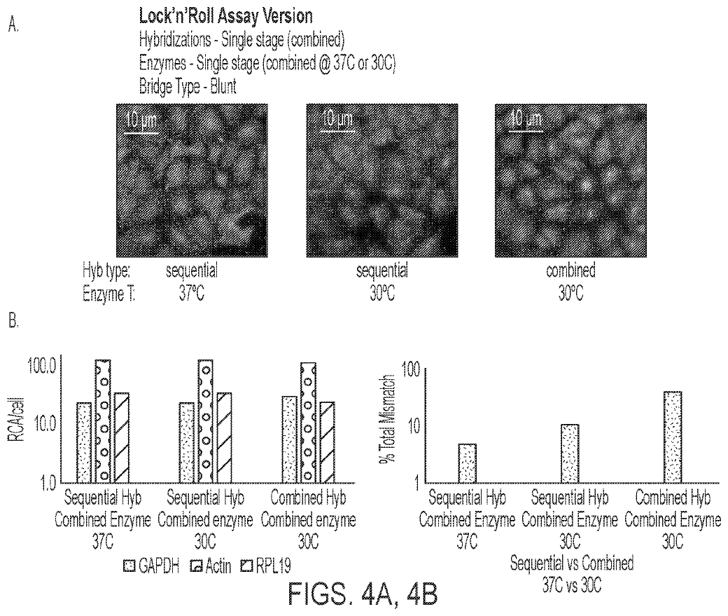

An experiment was performed to compare all 3 versions of the Hemi-bridge versus the Bridge Standard primer. In this experiment, the Bridge Standard primer and the Hemi-bridge primers were added in a second step after the annealing of the LnR ligation probes (corresponding to workflow of FIG. 2A). The results of the experiment, provided in FIG. 3, indicate that the Hemi-bridge system was not less efficient versus the Bridge Standard format. Of the three Hemi-bridge formats, the Bridge Blunt format seemed to perform slightly better. Off target ligations (mismatched LnR ligation probe sets) were also quantified for each condition and found not to differ substantially among any of the bridge formats. FIG. 3A shows the intracellular localization of the detected LnR RCA products. The per-cell quantifications of the images and off target ligations are provided in FIG. 3B.

### Discussion

In summary, a streamlined method is provided to perform the Lock'n'Roll assay, which involves the use of a Hemi-bridge primer format. Hemi-bridge formats enable the simultaneous hybridization of LnR ligation probes and bridge primer. This single oligonucleotide hybridization step can be followed by a combined ligase (e.g. Rnl2 and T4 DNA ligase and or a single stranded DNA ligase) and Phi29 polymerase step. The resulting streamlined protocol minimizes hands on time, without sacrificing assay performance. Streamlined Lock'n'Roll assays can be used for low to highly multiplexed spatial RNA analyses. LnR ligation probes can be designed to distinguish even closely related RNA molecules and the assay can likely be combined with other complementary-omics techniques including MERFISH and antibody staining assays when desired.

### Example 2: Demonstration of LISH-LnR with Readout Via Mass Spectrometry Tagged Oligonucleotide Probes

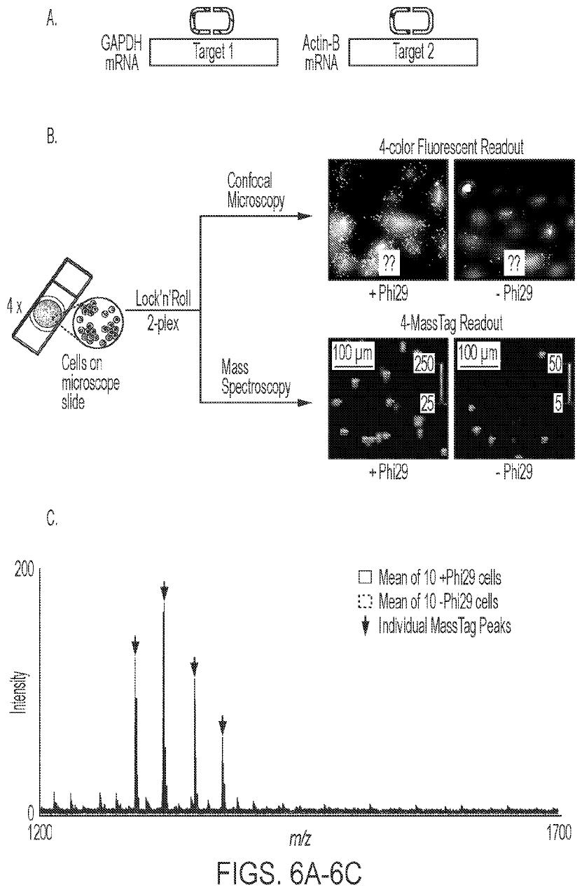

Two Ligation In Situ Hybridization Lock'n'Roll (LISH-LnR) probe sets were prepared as generally depicted in FIG. 6A. Each probe set is composed of a bipartite probe design as described in Example 1 above. Ligating target specific sequences of the bipartite probes and then the bridge binding sequences (whether with the hemi-bridge or full bridge design) creates a circular DNA molecule with the bridge primer also enabling amplification of the circularized sequence via rolling circle amplification (RCA). The readout probe binding sequences can then be detected by oligonucleotide probes conjugated either to fluorescent molecules or mass spectrometry tags.

A549 cells were cultured on a microscope slide and then fixed with 4% paraformaldehyde for 15 minutes and then prepared for LISH-LnR analysis. Two sets of slides were prepared, one for fluorescence imaging and one for imaging mass spectrometry. Each set contained all the components for a full LISH-LnR reaction or left out the Phi29 enzyme as a negative control. In the absence of Phi29, amplification of a circularized probe set is unable to occur. In the top images, we confirmed that RCA was indeed created, and could be imaged as expected by fluorescently conjugated oligonucleotide readout probes. Fluorescent signals were absent from the −Phi29 negative control as expected We observed the correct two colors colocalized with minimal incorrect colocalization of colors, which would arise from mismatched probe set ligations. Zoomed out imaging mass spec data collected from the cells, with or without Phi29 inclusion, is shown in FIG. 6B.

Peaks at the expected m/z were observed and these peaks were significantly higher in the cells that were treated with the LISH-LnR enzyme mix containing Phi29, versus the −Phi29 negative control. Quantification of the mass spectrometry tags corresponding to the images are shown in FIG. 6C. These data indicate that indeed LISH-LnR amplicon can be detected in situ via hybridization with oligonucleotide readout probes conjugated with mass-spectrometry tags, which in this case were released by photocleavage and detected using an imaging mass spectrometry instrument.

### REFERENCES

1. Crosetto N, Bienko M & van Oudenaarden A (2015) Spatially resolved transcriptomics and beyond. Nat Rev Genet 16, 57-66.
2. Lee J H (2017) Quantitative approaches for investigating the spatial context of gene expression. Wiley Interdiscip Rev Syst Biol Med 9, e1369.
3. Cheow L F, Courtois E T, Tan Y, Viswanathan R, Xing Q, Tan R Z, Tan D S, Robson P, Loh Y H, Quake S R et al. (2016) Single-cell multimodal profiling reveals cellular epigenetic heterogeneity. Nat Methods 13, 833-836.
4. Rozenblatt-Rosen O, Stubbington M J T, Regev A & Teichmann S A (2017) The human cell atlas: from vision to reality. Nature 550, 451-453.
5. Saper. C. B. (2009). A guide to the perplexed on the specificity of antibodies Journal of Histochemistry & Cytochemistry, 57(1), 1-5.
6. Belzil, V. V., Gendron, T. F., & Petrucelli, L. (2013). RNA-mediated toxicity in neurodegenerative disease. Molecular and Cellular Neuroscience, 56, 406-419.
7. O'Rourke, J. R., & Swanson, M. S. (2009). Mechanisms of RNA-mediated disease. Journal of Biological Chemistry, 284(12), 7419-7423.
8. Raj A, van den Bogaard P, Rifkin S A, van Oudenaarden A & Tyagi S (2008) Imaging individual mRNA molecules using multiple singly labeled probes. Nat Methods 5, 877-879.
9. Lubeck E, Coskun A F, Zhiyentayev T, Ahmad M & Cai L (2014) Single-cell in situ RNA profiling by sequential hybridization. Nat Methods 11, 360-361.
10. Shah S, Lubeck E, Zhou W & Cai L (2017) seqFISH accurately detects transcripts in single cells and reveals robust spatial organization in the hippocampus. Neuron 94, 752-758.e1.
11. Chen K H, Boettiger A N, Moffitt J R, Wang S & Zhuang X (2015) RNA imaging. Spatially resolved, highly multiplexed RNA profiling in single cells. Science 348, aaa6090.
12. Lee J H, Daugharthy E R, Scheiman J, Kalhor R, Yang J L, Ferrante T C, Terry R, Jeanty S S, Li C, Amamoto R et al. (2014) Highly multiplexed subcellular RNA sequencing in situ. Science 343, 1360-1363.
13. Smith, A. M., Heisler, L. E., St. Onge, R. P., Farias-Hesson, E., Wallace, I. M., Bodeau, J., . . . & Nislow, C. (2010). Highly-multiplexed barcode sequencing: an efficient method for parallel analysis of pooled samples. Nucleic acids research, 38(13), e142-e142.
14. Klein A M, Mazutis L, Akartuna I, Tallapragada N, Veres A, Li V, Peshkin L, Weitz D A & Kirschner M W (2015) Droplet barcoding for single-cell transcriptomics applied to embryonic stem cells. Cell 161, 1187-1201.
15. Stahl P L, Salmen F, Vickovic S, Lundmark A, Navarro J F, Magnusson J, Giacomello S, Asp M, Westholm J O, Huss M et al. (2016) Visualization and analysis of gene expression in tissue sections by spatial transcriptomics. Science 353, 78-82.
16. Tang F, Barbacioru C, Wang Y, Nordman E, Lee C, Xu N, Wang X, Bodeau J, Tuch B B, Siddiqui A et al. (2009) mRNA-Seq whole-transcriptome analysis of a single cell. Nat Methods 6, 377-382.
17. Larman, H. B., Scott, E. R., Wogan, M., Oliveira, G., Torkamani, A., & Schultz, P. G. (2014). Sensitive, multiplex and direct quantification of RNA sequences using a modified RASL assay. Nucleic acids research, 42(14), 9146-9157.
18. Credle, J. J., Itoh, C. Y., Yuan, T., Sharma, R., Scott, E. R., Workman, R. E., . . . & Miller, H. (2017). Multiplexed analysis of fixed tissue RNA using Ligation in situ Hybridization. Nucleic acids research, 45(14), e128-e128.

1. A method of detecting and localizing target nucleic acid sequences in situ, comprising:
(a) contacting a biological sample comprising a target nucleic acid sequence with a composition comprising (i) at least one target-specific ligation probe set and hemi-bridge oligonucleotides, (ii) at least one ligase and (iii) a strand-displacing polymerase;
(b) incubating the reaction mixture of step (a) under conditions that permit hybridization of the at least one ligation probe set to the target sequence present in the biological sample;
(c) circularizing the probe set and amplifying the circularized probe by strand-displacement amplification; and
(d) detecting and localizing target nucleic acid sequences in situ.

2. The method of claim 1, wherein the hemi-bridge oligonucleotides and the at least one ligation probe set are annealed prior to contacting the biological sample.

3. The method of claim 1, wherein the hemi-bridge oligonucleotides and the at least one ligation probe set are annealed simultaneously when contacting the sample.

4. The method of claim 1, wherein the hemi-bridge oligonucleotides comprise an overhanging 5′ end and a 3′ recessed end.

5. The method of claim 1, wherein the hemi-bridge oligonucleotides comprise a recessed 5′ end and a 3′ overhanging end.

6. The method of claim 1, wherein the hemi-bridge oligonucleotides comprise blunt 5′ and 3′ ends.

7-27. (canceled)

28. The method of claim 1, wherein the target nucleic acid sequence is a viral RNA, a bacterial RNA, a fungal RNA, a nematode RNA, a human RNA, a non-human mammal RNA, a nonmammalian animal RNA, a synthetic RNA or combinations thereof.

29. A kit comprising:
(i) at least one target-specific ligation probe set; and
(ii) hemi-bridge oligonucleotides.

30. The kit of claim 29 wherein the kit comprises a plurality of probe sets.

31. The kit of claim 29, further comprising at least one ligase, a strand-displacing polymerase or the combination thereof.

32. A method of detecting and localizing target nucleic acid sequences in situ, comprising:
(a) contacting a biological sample comprising a target nucleic RNA sequence with a composition comprising (i) at least one target-specific ligation probe set and an oligonucleotide primer that binds to one ligation probe, (ii) at least one double strand DNA or RNA ligase and one single strand DNA ligase and (iii) a strand-displacing polymerase;
(b) incubating the reaction mixture of step (a) under conditions that permit hybridization of the at least one ligation probe set to the target sequence present in the biological sample;
(c) circularizing the probe set and amplifying the circularized probe by strand-displacement amplification; and
(d) detecting and localizing target nucleic acid sequences in situ.

33. The method of claim 32, wherein the oligonucleotide primer and the at least one ligation probe set are annealed prior to contacting the biological sample.

34. The method of claim 32, wherein the oligonucleotide primer and the at least one ligation probe set are annealed simultaneously when contacting the sample.

35. The method of claim 32, wherein the oligonucleotide primer anneals to the 5′ donor ligation probe.

36-60. (canceled)

61. The kit of claim 29 wherein the kit comprises a plurality of probe sets.

62. The kit of claim 29, further comprising at least one ligase, a strand-displacing polymerase or the combination thereof.
